(19) United States
(12) Patent Application Publication
(10) Pub. No.: US 2013/0009779 A1
(43) Pub. Date: Jan. 10, 2013

(54) METHOD FOR DETERMINING THE ACTIVITY OF THE PARASYMPATHETIC NERVOUS SYSTEM AND/OR THE SYMPATHETIC NERVOUS SYSTEM OF THE AUTONOMIC NERVOUS SYSTEM OF A LIVING BEING

(76) Inventors: Werner Wittling, Neunkirchen (DE); Ralf Arne Wittling, Neunkirchen (DE)

(21) Appl. No.: 13/635,248

(22) PCT Filed: Mar. 18, 2011
(86) PCT No.: PCT/DE11/00291

§ 371 Date: Sep. 14, 2012

(30) Foreign Application Priority Data
Mar. 19, 2010 (DE) 10 2010 012 196.7
Aug. 11, 2010 (DE) 10 2010 034 055.3

(51) Int. Cl. A61B 5/0456; G08B 23/00; A61B 5/0402

(52) U.S. Cl. 340/576; 600/509; 600/510; 600/521

(57) ABSTRACT

The invention relates to a method for determining the activity of the parasympathetic nervous system or of the sympathetic nervous system of the autonomic nervous system of a living being, in particular a human being, wherein a feature of the condition of the living being is determined, and the activity is determined from the feature of the condition. According to the invention, the activity of the parasympathetic nervous system and/or of the sympathetic nervous system is determined dependent on time. Advantageously, the feature of the condition is a series of heartbeats of the living being, and the activity is determined by analyzing the time intervals between the heartbeats. For this purpose, the heart rate of the living being is preferably measured, and the heartbeat is determined using first positive deflections of a ventricular stimulus (R deflection). The invention further relates to a device for carrying out the method.

The invention concerns a method for determining the activity of the parasympathetic nervous system and/or the sympathetic nervous system of the autonomic nervous system of a living being, especially a human being, in which a feature of the condition of the living being is determined and recorded, and the activity is determined from this feature of the condition.

The physical state of a living being, e.g., its health, fitness or a stress state, is determined by, among other things, its ability to adapt to stresses and to withstand them. This adaptation occurs via the autonomic nervous system, which regulates the heart rate, respiration, blood pressure, digestion and metabolism as a function of the given stress. A division of the autonomic nervous system whose activation promotes performance readiness and responsiveness and the activation of energy reserves is known as the sympathetic nervous system. The activation of another division of the autonomic nervous system promotes rest and recovery and energy storage. This division is known as the parasympathetic nervous system, which acts as an antagonist to the sympathetic nervous system. Numerous studies have shown that the activity of the sympathetic nervous system and the parasympathetic nervous system can be used to assess the functional capacity of human beings. Since effects of activities of the sympathetic nervous system or parasympathetic nervous system can be associated, e.g., with variations of the time intervals between heartbeats, their measurement can be used to determine the given activity.

In a method of the aforementioned type that is well known through wide use, an electrocardiogram (EKG) of a person at rest is recorded over a period of several minutes. The time intervals are then analyzed by fast Fourier transform (FFT), and a frequency spectrum is constructed. A low-frequency region of this frequency spectrum, namely, 0.04 to 0.15 Hz, can be associated with the activity of the sympathetic nervous system, since the influence of the sympathetic nervous system on the heartbeat occurs in a relatively slow rhythm. A region of higher frequency, namely 0.15 to 0.4 Hz, is associated with the activity of the parasympathetic nervous system, which influences the heartbeat in a faster rhythm. By analyzing the frequency spectrum, the activities of the sympathetic nervous system and the parasympathetic nervous system relative to each other can be determined, and conclusions can be drawn about the physical and mental well-being of the individual.

In another well-known method that is used to determine fitness, a measurement is made of the amount of time needed for the pulse rate of the individual to return to a previously determined base rate after physical exercise (pulse recovery time). However, this widely used method is inexact.

More exact but also very expensive methods are lactate performance diagnosis and oxygen saturation analysis.

The objective of the invention is to develop a method with which the physical state of the individual can be evaluated quickly and simply.

In accordance with the invention, this objective is achieved by determining the activity of the parasympathetic nervous system and/or the sympathetic nervous system as a function of time.

The method of the invention makes it possible for changes in the activity of the parasympathetic nervous system and/or the sympathetic nervous system provoked by stimulation, for example, physical exercise, therapeutic, drug and/or electrical stimulation, psychic stress and/or pain, to be associated with specific points in time.

From this it is possible to draw conclusions about physical regeneration capacity and thus about the physical condition of the individual, especially his health or physical functional capacity.

It is advantageous to use a series of heartbeats of the individual as the feature of his condition.

The activity of the parasympathetic nervous system and/or the sympathetic nervous system can be determined by analyzing the time intervals between the heartbeats.

It is advantageous to record an electrocardiogram and to determine the heartbeat, preferably on the basis of the first positive deflections of ventricular stimulation (R deflection). Preferably, false signals of the measurement, caused, for example, by patient movement, are corrected. Alternatively, the heartbeat can be determined by picking up the pulse optically or mechanically.

In a refinement of the method, the time intervals, especially their variations, can be analyzed by the complex demodulation method (CDM).

This is a nonlinear rime range method for a time series analysis. While the aforementioned fast Fourier transform only allows determination of superposed frequencies from measurement over a prolonged period of time, with CDM it is also possible to analyze unsteady and discontinuous signals. The amplitude and the phase of a frequency component as a function of time are obtained as the result.

A time series X(t) can be represented as follows:

Xₜ=Aₜ*cos[f₀t+Pₜ] (1)
where Aₜ=slowly changing amplitude
   Pₜ=slowly changing phase

At a know frequency f₀, (1) can be substituted with the Euler equation:

Xₜ=(1/2)Aₜ {exp[i(f₀t+Pₜ)]+exp[−i(f₀t+P₁)]}

If Yₜ=Xₜ 2 exp(−if₀t),

then Yₜ=Aₜ[exp(iPₜ)+exp(−i{2f₀t+Pₜ})]

After a low-pass filtering of Yₜ, we obtain

yₜ=(1/2)Aₜ exp(iPₜ) where Aₜ=2|yₜ|

Pₜ=tan⁻¹[imag(h)/real(h)] where h=yₜ/|yₜ|

Viewed from the frequency range, the frequency band to be analyzed is shifted to zero and then low-pass filtered. The variation of the amplitude of the complex demodulation then reproduces the intensity of the signal around the frequency f₀. The variation of the phase describes the relative frequency deviation from the frequency f₀.

While the determination of only the activity of the parasympathetic nervous system, which is determined on the basis of frequencies of 0.15 to 0.4 Hz determined by the method of complex modulation and the amplitudes associated with these frequencies, is adequate for obtaining a sufficiently valid result, alternatively or additionally, the activity of the sympathetic nervous system can be determined, preferably on the basis of the frequencies of 0.04 to 0.15 Hz and the amplitudes associated with these frequencies. A reaction of the sympathetic nervous system to the stimulation can be determined and related to the activity of the parasympathetic nervous system.

The determination of activities at frequencies of 0.003 to 0.04 Hz is also conceivable. This allows even more exact evaluation.

In addition to FFT and CDM, it is also conceivable for the analysis to be carried out by means of the Hilbert transform, in-phase and in-quadrature filtering (filtering with ideal band pass and with the aid of quadrature modulation), phase-locked loop demodulation (demodulation with the aid of a PLL circuit), or peak amplitude and zero-crossing detection (determination of extreme values and zero crossings).

The activity of the parasympathetic nervous system and/or the sympathetic nervous system can be determined in a period of time before, during and/or after stimulation of the individual. However, in an especially preferred embodiment of the invention, it is determined by analyzing the time intervals within a period of time following a change in the stimulation, preferably a termination or reduction of the stimulation. This period of time is preferably at least 40 seconds. The reduction of the stimulation can consist, for example, in the case of physical exercise, in a period of relatively great output, for example, >300 W, being followed by much lower output, for example, <70 W, or no output at all.

It has been found that physical condition can be assessed on the basis of the change in activity of the parasympathetic nervous system that follows the change in stimulation. In addition, by repeating the method of the invention at greater intervals of time, e.g., regularly after several days, it is possible to assess the development of physical condition, for example, after taking drugs or other substances, or fitness, for example, after training with a certain training method. Results of the determination of the activity of the parasympathetic nervous system and/or the sympathetic nervous system are advantageously evaluated with consideration of the age of the individual and/or they can be evaluated with reference to normal values.

In an especially preferred embodiment of the invention, the stimulation is carried out as a function of the activity of the parasympathetic nervous system and/or the sympathetic nervous system. In this way, the duration and/or intensity of the stimulation can be adapted to the response of the individual to the stimulation. The interaction between the stimulation and the activity of the parasympathetic nervous system and/or the sympathetic nervous system can be used in making the evaluation.

It is advantageous to end the stimulation when the parasympathetic nervous system and/or the sympathetic nervous system shows a certain activity. It is conceivable that the stimulation can be ended when the given activity during a certain period of time, preferably during a period of 10 seconds, has a certain value. Preferably, the stimulation is ended when the parasympathetic nervous system is not active during this period of time.

Alternatively, the stimulation can be carried out upon expiration of a predetermined period of time, with the individual preferably being stressed by physical exercise on an ergometer. In this connection, the work output to be produced is preferably increased incrementally, in a way that is well known from exercise electrocardiography, until a certain maximum work load has been reached.

The invention also concerns equipment for determining the activity of the parasympathetic nervous system and/or the sympathetic nervous system of the autonomic nervous system of a living being, especially a human being, which includes a device for determining and/or recording a feature of the condition of the living being and a computer connected to this device for determining the activity from the feature of the condition of the living being.

In equipment of this type, which is already widely used, the measurements recorded by the EKG machine are transmitted to the computer, and after the measurement, software is used to analyze the measurement results by means of the fast Fourier transform, and, as described at the beginning, the frequency spectrum is associated with the activity of the sympathetic nervous system or the parasympathetic nervous system.

In accordance with the invention, the computer is provided for carrying out a time-dependent determination of the activity of the parasympathetic nervous system and/or the sympathetic nervous system, preferably by means of a suitable program.

The equipment is suitable for evaluating the physical condition of the individual, e.g., his health, his fitness, or a state of stress.

Advantageously, the means for determining the feature of the condition of the individual is a device for measuring his heartbeats, preferably a heart rate monitor or an EKG machine.

In one modification of the invention, the computer is designed to determine the activity from the time intervals between the heartbeats, preferably from information acquired from the EKG machine.

In one modification of the invention, the computer is designed to determine the activity by the method of complex demodulation (CDM), preferably on the basis of frequencies of 0.15 to 0.4 Hz and/or 0.04 to 0.15 Hz and of amplitudes associated with these frequencies.

In addition, the computer can be designed to determine activities at frequencies of 0.003 to 0.04 Hz.

It is advantageous for the equipment to include a device for stimulation of the individual, preferably an ergometer, which is preferably connected to the computer. The physical work performed by the individual can be determined with the ergometer and/or an amount of physical work to be performed can be preassigned to the individual. The data measured with the ergometer can be transmitted to the computer, and the computer is possibly set up to control the ergometer and especially to adjust the amount of physical work to be performed.

In a preferred embodiment, the device for providing stimulation and especially the intensity of the stimulation can be controlled as a function of the activity of the parasympathetic nervous system and/or the sympathetic nervous system. In particular, it is possible to adapt the physical work to be performed to the activity of the parasympathetic nervous system.

In one modification of the invention, the computer is designed to determine the activity of the parasympathetic nervous system and/or the sympathetic nervous system from the measurement of the feature of the condition of the individual during a period of time following termination of the stimulation of the individual. This period of time preferably lasts at least 40 seconds.

The computer can advantageously be a mobile device, e.g., a laptop or a device that can be worn on the body, such as a smart phone, or it can be of a sufficiently small size that it can be attached to the body, e.g., like a wrist watch. The heartbeats can be determined in an already well-known way with a chest strap that determines the heartbeats by means of two integrated skin electrodes. It is advantageous to connect the chest strap to the computer in such a way that information determined by means of the chest strap can be transmitted to the computer, e.g., by radio.

In a modification of the invention, the method and equipment described above are used to carry out a biofeedback process, in which the activity of the parasympathetic nervous system and/or the sympathetic nervous system of the individual is measured, and the measurement results are continuously displayed, e.g., on a display screen, so that the individual can watch them. The advantage of this method is that allows the individual to learn to control the activity of the parasympathetic nervous system and/or the sympathetic nervous system, e.g., by breathing technique, autogenic training or the like.

Furthermore, the method and equipment can be used to determine an effect and/or influence on the physical condition of the individual produced by the administration of drugs or other substances, e.g., caffeine, alcohol or nicotine, or to determine an effect of relaxation techniques, e.g., breathing therapy, acupuncture, yoga or progressive muscle relaxation, effects of stress factors, or reactions to advertising messages, especially for marketing research purposes.

In addition, the method and equipment can be used to detect and possibly determine an infection, hypertension, a change in cholesterol, or an inflammatory process.

Another possible application is continuous evaluation of the ability of an individual to drive a motor vehicle or operate machinery.

The method or the equipment can also be used to control parameters of an environment of an individual, e.g., lighting intensity, coloration or sounds, according to the physical state of the individual. For this purpose, the equipment is advantageously connected with a control device by which the parameters can be adjusted.

Finally, the equipment or the method can be used for controlling interactive computer games or television or video films.

The invention will now be explained in greater detail with reference to specific embodiments and to the accompanying drawings related to these specific embodiments.

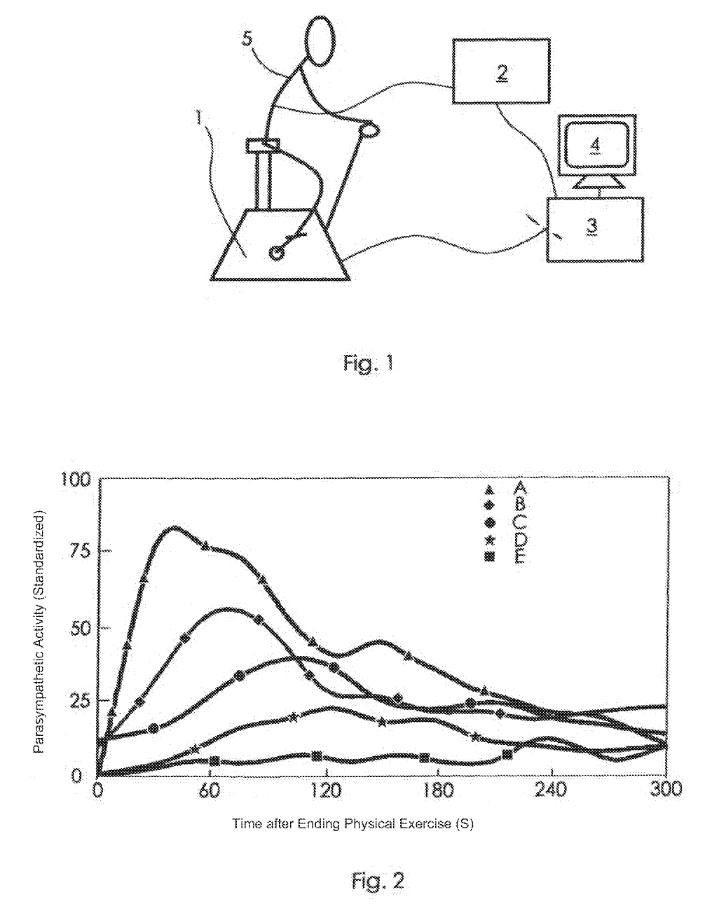

FIG. 1 is a schematic representation of the equipment of the invention.

FIG. 2 is a graph that shows the results of measurements by the method of the invention.

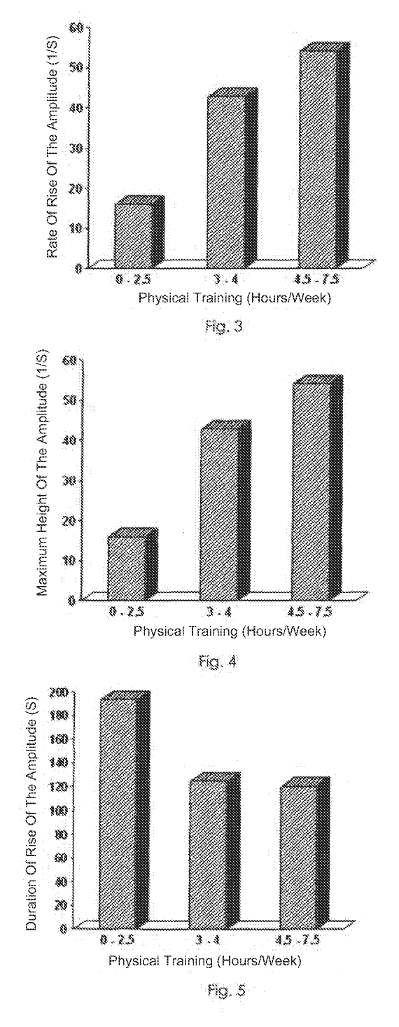

FIGS. 3 to 5 are graphs of results of a study in which the method of the invention was used.

FIG. 1 shows equipment of the invention, which comprises a bicycle ergometer 1, an EKG machine 2, and a computer 3 with a monitor 4. A person 5 is shown sitting on the bicycle ergometer for the purpose of illustration.

The computer 3 is connected with the bicycle ergometer 1 in such a way that the physical work performed by the person 5 is displayed on the monitor 4 and can be stored as a function of time. In addition, the computer 3 can be used to preassign an amount of work to be performed by the person 5. For this purpose, the computer 3 can also adjust the work to be performed on the bicycle ergometer 1, for example, by increasing the resistance of the pedals of the bicycle ergometer 1.

Other types of ergometers can be used as alternatives to the bicycle ergometer, for example, cross trainer machines, rowing ergometers, treadmills and home exercise equipment.

The EKG machine 2 includes electrodes arranged on the body surface of the person 5 and is likewise connected to the computer 3. An electrocardiogram recorded with the EKG machine 2 is transmitted to the computer 3 and stored there.

Alternatively, the equipment of the invention can include a device for monitoring the heart rate optically and/or mechanically. Devices of this type include especially such well-known devices as a chest strap or sensors mounted in the hand grips of the bicycle ergometer.

It is possible for the EKG machine 2 or the aforementioned devices for monitoring the heart rate and the computer 3 and monitor 4 to be integrated in the bicycle ergometer or other specified types of ergometers.

When the method of the invention is carried out, the computer runs a program that is designed to store and evaluate measurement results and to display the measurement results and evaluations. In addition, it guides the person 5 through the method.

First, as in the case of an exercise EKG, the person 5 produces an incrementally increasing work output by pedaling the bicycle ergometer 1. Within the first minute, 30 W are generated, and then the work output is increased minute by minute to 150 W, so that an output of 150 W is being produced within five minutes. At the end of the fifth minute, the person 5 stops pedaling and remains at rest while his heartbeat is recorded. Alternatively, the person 5 can continue to pedal at a much lower work output of <70 W and preferably <50 W.

Alternatively, the work load can also be increased continuously.

In addition, the duration of the exercise can be varied, e.g., on the basis of an estimate of the physical functional capacity of the person 5.

To obtain a valid result, at least 40 seconds must be recorded. However, valuable information can be obtained especially from measurements of the first 300 seconds.

By means of the aforesaid program, the measurement results are stored, the measurement result is tested for measurement errors that may have occurred, and these errors are corrected. An analysis is then undertaken by the method of complex demodulation (CDM). Frequencies superposed in the rhythm of the heartbeats and amplitudes associated with these frequencies are determined and assigned to an activity of the parasympathetic nervous system that lies in the frequency range of 0.15 to 0.4 Hz and possibly to an activity of the sympathetic nervous system that lies in a frequency range of 0.04 to 0.15 Hz. The behavior of the given activity as a function of time can be graphically represented on the monitor 4, as shown, for example, in the graph in FIG. 2. Besides the results of the analysis, the software can indicate normal values for the physical characteristics of the person 5, in relation to which the person's fitness can be assessed.

In the graph according to FIG. 2, the activity of the parasympathetic nervous system, as determined by the method described here, is graphed for varyingly well-trained persons A, B, C, D and E as a function of time after termination of the physical exercise described above. Person A participates in sports activity for more than 9 hours per week, person B for 5-9 hours, person C for 2-5 hours, person D for 0.5-2 hours, and person E for less than 0.5 hours. As the graph shows, there are clear differences between the differently well-trained persons with respect to the activity of the parasympathetic nervous system as a function of time. Thus, the highly physically trained person A shows a greater rate of rise and a greater maximum amplitude than the other test subjects. The graph shows that the less well trained the persons are, the slower the rate of rise, the lower the maximum amplitude, and the longer the period of time during which the amplitude rises.

In a study with 37 test subjects with an average age of 26.22 years, of whom 19 where female and 18 were male, it was found that the method of the invention was able to determine significant differences between the test subjects as a function of their weekly physical activity. The test subjects were divided into three groups. The first group participated in sports activity for 0-2.5 hours per week, the second for 3-4 hours per week, and the third for 4.5-7.5 hours per week.

As FIG. 3 shows, the rate of rise described above is significantly lower in the test subjects who participated in sports activities for only 0-2.5 hours per week than that of the other test subjects. Moreover, the rate of rise for the test subjects that had 4.5-7.5 hours of sports activity per week is higher than that of the test subjects that had 3-4 hours of sports activity per week.

Similar differences are found for the maximum amplitude level of the parasympathetic activity, which is shown in FIG. 4.

The graph in FIG. 5 shows that the period of time during which the amplitude rises is significantly higher for persons with the least amount of sports activity than for the persons with greater training.

The method of the invention is suitable not only for a one-time evaluation of the physical condition of a person but also and especially for monitoring a person's condition over an extended period of time, in which case the method would be carried out at regular intervals and the results compared with previous results. Insufficient activation of the parasympathetic nervous system could thus give an early indication of the onset of cardiovascular diseases.

The method also offers the advantageous possibility, for example, of evaluating the effect of drugs or therapies or the effectiveness of a certain training method.

It is also conceivable that the method could be used for evaluating the health of animals. This method could be of interest especially for testing the fitness of competitive animals, such as horses, dogs, or camels. The equipment of the invention can be adapted to the individual animal species.

In another embodiment of the invention, the activity of the parasympathetic nervous system and possibly the sympathetic nervous system of the person 5 is already determined during and possibly even before the person first starts exercising on the bicycle ergometer 1. The activity of the parasympathetic nervous system before or at the beginning of exercise and its response to the physical load, especially its change and/or the time until a certain activity is reached, especially as a function of the work output produced or to be produced, is a measure of the physical fitness of the person 5. One measure, for example, is the rate of decline of the parasympathetic nervous system activity, measured as the quotient of the activity before or at the beginning of exercise and the length of time until inactivity of the parasympathetic nervous system is reached.

Furthermore, information obtained in this way for the activity of the parasympathetic nervous system during the initial exercise can be used to control the level of work to be performed by the person 5.

For example, the incrementally increasing work to be performed on the bicycle ergometer 1 can be ended exactly when the parasympathetic nervous system has a certain activity over a period of e.g., 10 seconds, or is no longer active. The exercise of a less well trained person is then ended earlier than that of a person in better training.

Alternatively, it is possible for the work to be performed on the bicycle ergometer 1 to be controlled as a function of the activity of the parasympathetic nervous system measuring during exercise. This makes it possible to ensure that for the individual being tested, the parasympathetic nervous system has a certain activity as a function of time during the exercise phase. The work to be performed by a well-trained person is then increased more rapidly than the work to be performed by a less fit person.

The initial exercise can be followed by measurement at rest or at a low work load as described above.

When the exercise is controlled as a function of the activity of the parasympathetic nervous system, it becomes possible to adjust the exercise phase to the individual person who is being tested and to create comparable conditions for the measurement at rest or at a lower work load.

To make it possible to control the amount of work to be performed as a function of the activity of the parasympathetic nervous system, the computer 3 is designed in such a way that the work to be performed can be directly controlled on the basis of the evaluations of the measurement results, i.e., on the basis of the activity of the parasympathetic nervous system determined from the measurement results. The computer 3 determines the activity of the parasympathetic nervous system and controls the bicycle ergometer 1 as a function of the measurement, i.e., during the time of the work load on the person 5 on the bicycle ergometer 1.

1-15. (canceled)

16. A method for determining activity of the parasympathetic nervous system and/or the sympathetic nervous system of the autonomic nervous system of a living being, the method comprising the steps of: determining a feature of the condition of the living being is determined; and determining the activity from the feature of the condition, wherein the activity of the parasympathetic nervous system and/or the sympathetic nervous system is determined as a function of time.

17. The method in accordance with claim 16, wherein the feature of the condition of the living being is a series of heartbeats and that the activity is determined by analyzing time intervals between the heartbeats.

18. The method in accordance with claim 17, including making a heart rate measurement, with the heartbeat being determined based on first positive deflections of ventricular stimulation (R deflection).

19. The method in accordance with claim 18, including making the heart rate measurement by recording an electrocardiogram (EKG) of the living being.

20. The method in accordance with claim 17, including analyzing time intervals or time variations by a method of complex demodulation method (CDM).

21. The method in accordance with claim 16, including determining the activity of the parasympathetic nervous system based on frequencies of 0.15 to 0.4 Hz and amplitudes associated with these frequencies, and determining the activity of the sympathetic nervous system based on frequencies of 0.04 to 0.15 Hz determined by a method of complex demodulation and amplitudes associated with these frequencies.

22. The method in accordance with claim 18, including determining the activity in a period before, during and/or after stimulation of the living being.

23. The method in accordance with claim 22, including determining the activity after physical exercise, therapeutic, drug and/or electrical stimulation, psychic stress and/or pain.

24. The method in accordance with claim 22, including carrying out the stimulation as a function of the activity of the parasympathetic nervous system and/or the sympathetic nervous system.

25. The method in accordance with claim 22, including determining the activity by analyzing the time intervals within a period of time following a change in the stimulation.

26. The method in accordance with claim 25, wherein the period of time is 40 seconds.

27. The method in accordance with claim 25, wherein the change in the stimulation is termination or reduction of the stimulation.

28. The method in accordance with claim 16, wherein the living being is a human being.

29. Equipment for determining activity of the parasympathetic nervous system and/or the sympathetic nervous system of the autonomic nervous system of a living being, comprising: a device for determining a feature of the condition of the living being; and a computer connected to the device for determining the activity from the feature of the condition of the living being, wherein the computer is operative to determine the activity of the parasympathetic nervous system and/or the sympathetic nervous system as a function of time.

30. The equipment in accordance with claim 29, wherein the device is a device for measuring heartbeats of the living being.

31. The equipment in accordance with claim 29, wherein the computer is operative to determine the activity by a method of complex demodulation (CDM).

32. The equipment in accordance with claim 31, wherein the computer is operative to determine the activity from valves of the intervals between first positive deflections of a ventricle (R-R interval).

33. The equipment in accordance with claim 31, wherein the computer is operative to determine the activity by the method of complex demodulation (CDM) based on certain frequencies of 0.15 to 0.4 Hz and/or 0.04 to 0.15 Hz.

34. The equipment in accordance with claim 33, wherein the computer is operative to determine the activity based on amplitudes associated with the frequencies.

35. The equipment in accordance with claim 30, wherein the computer is operative to determine the activity from a series of the heartbeats within a period of time following a change in stimulation.

36. The equipment in accordance with claim 35, wherein the period of time is 40 seconds.

37. The equipment in accordance with claim 35, wherein the change is stimulation is a termination or reduction of the stimulation.

38. The method in accordance with claim 16, further including determining an effect and/or influence on a physical condition of an individual produced by administration of drugs or other substances, determining an effect of a relaxation treatment or an effect of stress factors, determining reactions to advertising messages, or detecting an infection, hypertension, a change in cholesterol, or an inflammatory process based on the determined activity of the parasympathetic nervous system and/or the sympathetic nervous system.

39. The method in accordance with claim 16, further including continuously evaluating the ability of an individual to drive a motor vehicle or operate machinery, controlling parameters of an environment of the individual, or controlling interactive computer games or television or video films based on the determined activity of the parasympathetic nervous system and/or the sympathetic nervous system.

40. The method in accordance with claim 39, wherein the environmental parameters are selected from the group consisting of lighting intensity, coloration or sounds in the environment.
